SEQUENCE LISTING (4 sequences)

SEQ ID NO: 1 - DNA, 25 bp, Artificial Sequence
  Synthesized
tcacattagtgttggagtaatagcc

SEQ ID NO: 2 - DNA, 22 bp, Artificial Sequence
  Synthesized
caccatacccttgctagaacct

SEQ ID NO: 3 - DNA, 101 bp, Artificial Sequence
  Synthesized
tctcttggtttatgggtattaaataacataattttttttttgatcaataattagttatccttgagcaatatacgacataaagtaaggttctagcaagggta

SEQ ID NO: 4 - DNA, 99 bp, Artificial Sequence
  Synthesized
tctcttggtttatgggtattaaataacataattttttttgatcaataattagttatccttgagcaatatacgacataaagtaaggttctagcaagggta

(19) United States
(12) Patent Application Publication
(10) Pub. No.: US 2025/0019776 A1
(43) Pub. Date: Jan. 16, 2025

(54) MOLECULAR MARKER OF GENE RELATED TO DOMINANT EARLY HEADING IN RICE MATERIAL CAPABLE OF EARLY HEADING WITHOUT DECREASING YIELD AND USE THEREOF

(71) Applicant: INSTITUTE OF CROP SCIENCES, CHINESE ACADEMY OF AGRICULTURAL SCIENCES, Beijing (CN)

(72) Inventors: Tianqing ZHENG, Beijing (CN); Linyun XU, Beijing (CN); Muhiuddin FARUQUEE, Beijing (CN); Jianlong XU, Beijing (CN); Binying FU, Beijing (CN); Xiuqin ZHAO, Beijing (CN); Zhikang LI, Beijing (CN)

(21) Appl. No.: 18/245,714

(22) PCT Filed: Sep. 17, 2020
(86) PCT No.: PCT/CN2020/115820

§ 371 Date: Mar. 16, 2023

(51) Int. Cl. C12Q 1/6895; A01H 1/04

(52) U.S. Cl. CPC C12Q 1/6895; A01H 1/045; C12Q 2600/13; C12Q 2600/156

(57) ABSTRACT

The present invention discloses a specific primer ZMEH_1 for identifying or assisting in identifying the heading date of rice materials and use thereof, and a breeding method of rice cultivars with early-heading without yield drag. The ZMEH_1 is a pair of PCR primers corresponding to the upstream and downstream of the fragment with sequence variation 1 of the rice Os03g0122600 gene between the late- heading parent MH63 and the early-heading parent DEH229. ZMEH_1 can explain about 86.0% of the phenotypic variation of the progeny from the MH63/DEH229 population, which meets the needs of industrial applications. DEH229 has the characteristic of early-heading without yield drag, and its progenies derived from various rice cultivars all continue to have the characteristic of early-heading without yield drag. The specific primer ZMEH_1 can be used to quickly screen out the materials with early-heading characteristic among the progeny rice materials derived from DEH229, and carry out rapid breeding and utilization to promote the breeding process of early-maturing rice without affecting the yield.

### INCORPORATION-BY-REFERENCE OF MATERIAL IN THE ASCII TEXT FILE

The contents of the electronic sequence listing (SEQUENCELISTING.txt; Size: 1233 bytes; and Date of Creation: Mar. 16, 2023) is hereby incorporated herein by reference in its entirety.

### FIELD OF THE INVENTION

The present invention relates to molecular markers of dominant early-heading related genes in rice materials of early-heading without yield drag and use thereof in the field of biotechnology.

### BACKGROUND OF THE INVENTION

Rice (Oryza sativa L.) is a model crop, and the heading date (HD) shapes the differences between different cultivars; as the main food source for the global population, the grain yield (GY) is still a major goal of rice breeding. Therefore, understanding the genes related to heading date and yield, especially the pleiotropic genes, is of great application value. However, many pleiotropic genes reported at present often have phenotypes of increased yield and delayed heading date, which largely limits their direct application in breeding new cultivars. Early-heading without yield drag is one of the important strategies for crop cultivar improvement, and dominant carly-heading without yield drag has a more important application value.

The heading date of rice is controlled by complex multi-factors. Studies have found that carly-heading is mostly a recessive trait and is directly related to decrease in yield. The genetic mechanism of dominant carly-heading (DEH) is not yet fully understood. For example, the carly-heading trait of Zaoxian A may be controlled by some dominant genes, but there is no further report. The carly-heading characteristics of Kefeng A vary by corss (1); Zaoxian A (2) and H14 (3), which exhibit partial dominance, are both derived from the progeny of distant crosses; the indica maintainer line D64B was crossed with parents with far genetic distance, and its early-heading characteristics were more thorough (4). In addition, there are Lexiang 202B (5), Calose 76 (6) carrying sdl, and R1-8 and R1-2 (7) from IR20. Recently, an important genetic factor Ef-cd (early flowering-complete dominance) in 6442S-7 was cloned, 20 years after the first report on a pair of major genes in 6442S-7 (8). In addition, DEH materials are mostly derived from the background of sterile lines or their early-heading expression is partially dominant, so their breeding utilization is greatly limited.

References:

1.X.J. Deng, K.D. Zhou, R.D. Li, P. Li, G.S. Huang, and C. Ze, Genetic analysis of dominant earliness of rice genic male sterile line 6442S-7. Acta Genetica Sinica, 28 (2001) 628-634.

2.Y.L. Xiao, C.Y. Yu, J.G. Le, M.Z. Li, L. Jiang, and J.M. Wan, Genetic mechanism of the dominant earliness in Ke-Feng A, a new cytoplasmic male sterile line in rice. Chinese Journal of Rice Science, 23 (2009) 271-276.

3.Y.L. Xiao, C.Y. Yu, J.G. Le, M.Z. Li, L. Jiang, and J.M. Wan, Genetic mechanism of the dominant earliness in Ke-Feng A, a new cytoplasmic male sterile line in rice. Chinese Journal of Rice Science, 23 (2009) 271-276.

4.X.L. Li, Y.S. Jiang, P.R. Wang, and X.J. Deng, Genetic analysis and gene mapping of dominant earliness of rice line H14. Chinese Journal of Applied & Environmental Biology, 14 (2008) 332-336.

5.Y.J. Yang, X.D. Wang, X.J. Wu, H. Y. Zhang, P. Zhang, and H.X. Zhao, The discovery, genetic analysis and gene mapping of earliness rice (Oryza sativa L.) D64B. Acta Genetica Sinica, 32 (2005) 495-500.

6.X.Z. Wang, Genetic analysis of earliness in some lines and molecular mapping of the gene controlling earliness in Le-Xiang202B of rice (Oryza sativa L.). Master. 2004: Sichuan Agricultural University.pp. 49.

7.K.S. Mckenzie, J.E. Board, K.W. Foster, and J.N. Rutger, Inheritance of heading date of an induced mutant for early maturity in rice (Oryza sativa L.). SABRAO Journal of Breeding and Genetics, 10 (1978) 96-102.

8.R.C. Yang, N.Y. Wang, K.J. Liang, and Q.H. Chen, Inheritance of early-maturing and semidwarfing mutants of rice. Application of Atomic Energy in Agriculture, (1986) 24-29.

### SUMMARY OF THE INVENTION

A technical problem to be solved by the present invention is how to identify or assist in identifying rice materials with early-heading without yield drag.

In order to solve the above technical problems, the present invention provides a pair of specific primers for identifying or assisting in identifying the heading date of rice materials.

The specific primers provided by the present invention for identifying or assisting in identifying the heading date of rice materials are ZMEH_1; the ZMEH_1 is a pair of PCR primers corresponding to the upstream and downstream of the fragment with sequence variation 1 of rice Os03g0122600 gene between MH63 and DEH229; the fragment with sequence variation 1 is ATTT or AT at position 1234469 on Os03g0122600 gene. The ZMEH_1 is two single-stranded DNAs with nucleotide sequences of SEQ ID No.1 and SEQ ID No.2, respectively.

The present invention further provides a method for assisting in identifying the heading date of rice using the ZMEH_1 marker by using the genomic DNAs of the late-heading parent MH63, the early-heading parent DEH229 and the progeny line to be identified as templates, respectively, using the PCR primer pairs that are formed by two single-stranded DNAs shown in SEQ ID No.1 and SEQ ID No. 2 to carry out PCR amplification, detecting an obtained PCR product, and determining the heading date of the progeny line to be identified according to the following method:

If the PCR amplification product of the progeny line to be identified is the same as that of the PCR amplification product of the late-heading parent MH63, then the progeny line to be identified is a late-heading line or a candidate for late-heading line; if the PCR amplification product of the progeny line to be identified is the same as that of the early-heading parent DEH229, the line to be identified is an early-heading line or a candidate for an early-heading line.

In the above method, the step of detecting an obtained PCR product is to detect the size of the PCR product by electrophoresis and/or to detect the sequence information of the PCR product by sequencing.

In the above method, the PCR amplification product of the late-heading parent MH63 is a single-stranded DNA with a nucleotide sequence as shown in SEQ ID No.3; the PCR amplification product of the early-heading parent DEH229 is a single-stranded DNA with a nucleotide sequence as shown in SEQ ID No.4.

In the above method, the progeny line to be identified is rice material derived from DEH229. The derivation is a new rice material obtained by conventional breeding. biotechnological breeding, asexual propagation, or a combination thereof, using DEH229 as a parent or one of the parents. The conventional breeding, includes common breeding methods such as self-breeding, crossbreeding, backcrossing, and combinations thereof. The biotechnological breeding includes known biotechnological breeding methods such as gene editing breeding, transgenic breeding, mutagenic breeding, double haploid breeding, molecular marker breeding, and combinations thereof.

In the above method, the early-heading is that the heading date under long-day conditions and short-day conditions is earlier than that of MH63 under the same conditions; there is no significant difference between the late-heading being a long-day condition or a short-day condition at heading date compared to MH63 at the heading date under the same conditions.

The present invention further protects a reagent or kit for identifying or assisting in

identifying the heading date of rice, the reagent or kit includes the specific primer ZMEH_1.

The specific primer ZMEH_1, the method and/or the use of the reagent or kit in identifying or assisting in identifying the heading date of rice materials belongs to the protection scope of the invention.

The specific primer ZMEH_1, the method and/or the use of the reagent or kit in rice breeding further belongs to the protection scope of the invention.

The present invention further protects a breeding method of rice cultivars with early-heading without yield drag, including the steps of using DEH229 as a parent or one of the parents to breed progeny, using the specific primer ZMEH_1 to select early-heading materials from the progeny for further breeding to obtain rice with early-heading without yield drag.

In the above method, the breeding of progeny is carried out by conventional breeding, biotechnological breeding, asexual propagation, or a combination thereof.

In the above method, the conventional breeding includes common breeding methods such as crossbreeding, backcrossing, and self-breeding.

The present invention further protects rice with carly-heading without yield drag produced by the breeding method.

The present invention developed a pair of specific primers ZMEH_1 for rapid differentiation of heading date of rice based on an insertional deletion (InDel) difference between MH63 and DEH229 in the rice Os03g0122600 gene. Experiments have proved that ZMEH_1can explain about 86.0% of the phenotypic variation of DEH229-derived progeny, which meets the needs of industrial application. The specific primer ZMEH_1 can be used to identify early-heading and late-heading by the size of the bands amplified by PCR, and this method is time-saving, labor-saving, simple compared to previous methods, and the result is accurate and reliable. It has been verified that DEH229 has the characteristic of early-heading without yield drag, and its progeny derived from various rice cultivars such as MH63, 9311 and R498 all continue to have the characteristic of early-heading without yield drag. The specific primer ZMEH_1 can be used to quickly screen out the materials with early-heading characteristics among the progeny rice materials derived from DEH229, and carry out rapid breeding and utilization, so as to promote the breeding process of early-maturing rice without affecting the yield.

### BRIEF DESCRIPTION OF THE FIGURE

FIG. 1 shows the genome comparison between MH63 and 3027 and the comparison of phenotypes under different sunshine conditions. The left side of the figure is the genome comparison, and the right side is the phenotype comparison. BJ is Beijing (long-day conditions), and SY is Sanya (short-day conditions).

FIG. 2 is a phenotype comparison chart between DEH229 and MH63, in which DEH229is DEH229, and Minghui 63 is MH63.

FIG. 3 is a graph comparing the genome variation of DEH229 and MH63, and the DEH in the figure is DEH229.

FIG. 4 shows the phenotypes of MH63 (P₁), DEH229 (P₂) and their progeny (F₁ and F₂), where the data of parents and F₁ individuals are collected in triplicate. a) and b) are the phenotypes of representative individuals in the field and pots, respectively. c) Phenotypic differences in other agronomic traits, including plant height (PH), panicle number (PN), seed setting rate (SF), thousand-grain weight (TGW), and yield (GY) among P₁, P₂, and F₁, in addition to rice shape. (White bar=1 cm); d) Distribution of heading date (DTH) in P₁, P₂, F₁ and F₂.

FIG. 5 shows the phenotypes of DEH229 crossed with two representative restorer lines 9311 and R498. a) Phenotypes of P₁=9311, P₂=DEH229, and their progeny (F₁ and F₂). b) Phenotypes of P₁=R498, P₂=DEH229, and their progeny (F₁ and F₂). c) 9311 (P₁), DEH229 (P₂), the difference between their F₁ and F₂ generations, including plant height (PH), panicle number (PN), seed setting rate (SF), grains per panicle (GNP), thousand-grain weight (TGW) and yield (GY). d) Differences between R498 (P₁), DEH229 (P₂), and their F₁ and F₂ generations; the traits are the same as those shown in FIG. c 2.

FIG. 6 is a genomic map of the loci associated with variation and heading date. a) G′ values of the MH63/DEH229 F₂ population detected by QTLseqr. b) Manhattan plot of heading variation in 3K-RG. c) Number of variants (SNPs and InDels) between DEH229 and MH63.

FIG. 7 is a haplotype analysis diagram of gene Os03g0122600. a) CDS, b) 2000bp promoter, c) comparison of phenotypic effects of haplotypes in the 5′UTR and d) 3′UTR regions.

FIG. 8 shows the positions of the six variants between MH63 and DEH229 on the MH63RS2 reference map for gene Os03g0122600.

FIG. 9 is the result of the determination of the progeny of the MH63/DEH229 population using InDel-labeled ZMEH_1 gels. In the figure, the samples in wells 3 and 65 are MH63, the samples in wells 4 and 66 are DEH229, and the samples in wells 5-64 are those with representative phenotypes where samples in wells 5-33 are early-heading lines, while samples in wells 34-64 are late-heading lines and samples in wells 1, 2, 67 and 68 are Ladder.

The Best Way to Practice the Invention

The present invention will be described in further detail below in connection with specific embodiments, and the given examples are only for clarifying the present invention, not for limiting the scope of the present invention. The examples provided below can be used as a guide for those skilled in the art to make further improvements and are not intended to limit the present invention in any way.

The experimental methods in the following examples are conventional methods unless otherwise specified. Unless otherwise specified, the materials and reagents used in the following examples can be obtained from commercial sources.

The indica restorer variety Minghui 63 (MH63 for short) is recorded in the non-patent literature “Ding Li, Qi Yongwen, Zhang Hongliang, Zhang Dongling, Wang Meixing, Li Zichao, Tang Shengxiang. Genetic diversity of Chinese three-line hybrid rice restorer line resources. Acta Agronomica Sinica: 2007. 33, 1567-1594”, the public can obtain it from the Institute of Crop Sciences, Chinese Academy of Agricultural Sciences to repeat the experiment of the present application, and it cannot be used for other purposes.

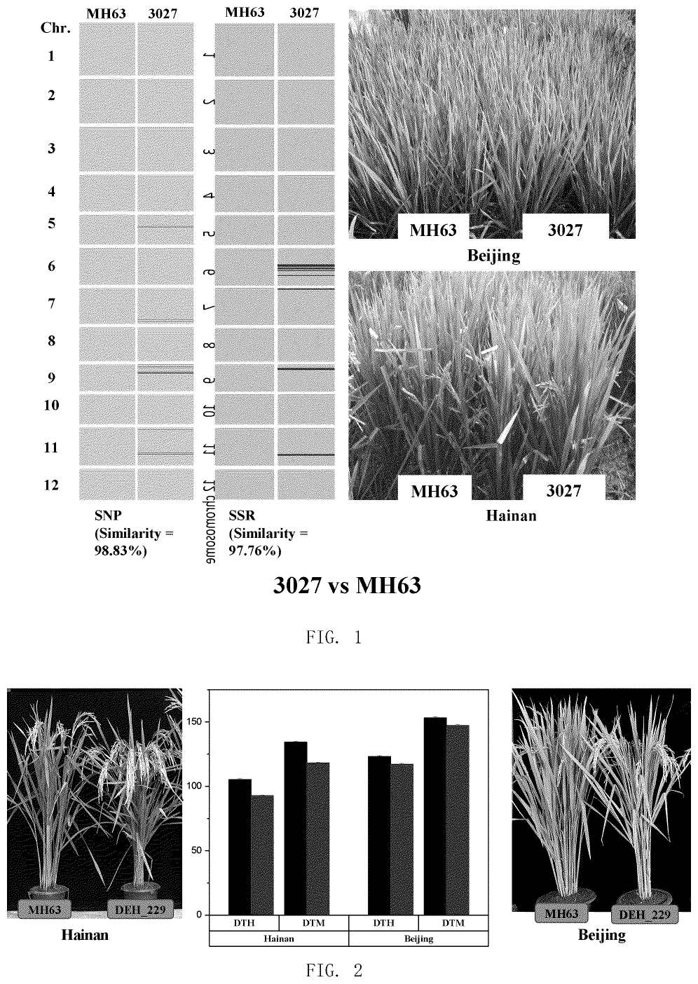

Rice 3027 is a near-isogenic line of MH63, with similar phenotypes, including heading date with MH63. The genome similarity between the two is as high as 98.83% (SNP) and 97.76% (SSR). See FIG. 1 for details. Rice 3027 is recorded in the non-patent reference “Zhang Qiang. Non-Mendelian Genetic Research of Rolling Leaf QTL Mapping and SSR Markers in Rice. Shenyang Agricultural University, 2016”, and the public can obtain it from the Institute of Crop Sciences, Chinese Academy of Agricultural Sciences to repeat the experiment of the present application, and it cannot be used for other purposes.

DEH229 is a rice-inbred variety obtained by crossing MH63 as the female parent and 3027 as the male parent, followed by continuous backcross selection. Rice variety DEH229 has applied for new plant variety rights in my country, which is recorded in the “Announcement on Application for Variety Rights on Jul. 1, 2020 (Total No. 126), Ministry of Agriculture and Rural Affairs, Jul. 1, 2020”, application Ser. No. 20/201,000376, announcement No. CNA031646E, announcement date July 1. 2020. The public can obtain DEH229 from the Institute of Crop Sciences, Chinese Academy of Agricultural Sciences to repeat the experiment of the present application, and it cannot be used for other purposes.

Rice 9311 is a commonly used indica restorer line, which is recorded in the non-patent reference “Wang Fangquan, Fan Fangjun, Xia Shijian, Zong Shouyu, Zheng Tianqing, Wang Jun, Li Wenqi, Xu Yang, Chen Zhihui, Jiang Yanjie, Tao Yajun, Zhong Weigong, Yang Jie. Interaction effect of rice photothermosensitive sterility gene tms5 and pms3. Acta Agronomica Sinica. 2020:03, 5-17”, the public can obtain it from the Institute of Crop Sciences, Chinese Academy of Agricultural Sciences to repeat the experiment of the present application, and it cannot be used for other purposes.

Rice R498 is an indica restorer line, which is recorded in the non-patent reference “Wang Fangquan, Fan Fangjun, Xia Shijian, Zong Shouyu, Zheng Tianqing, Wang Jun, Li Wenqi, Xu Yang, Chen Zhihui, Jiang Yanjie, Tao Yajun, Zhong Weigong, Yang Jie. Interaction effect of rice photothermosensitive sterility gene tms5 and pms3. Acta Agronomica Sinica, 2020:03, 5-17”, the public can obtain it from the Institute of Crop Sciences, Chinese Academy of Agricultural Sciences to repeat the experiment of the present application, and it cannot be used for other purposes.

### EXAMPLE 1

The rice materials in this example were planted under the conditions of long-day (Beijing major season) and short-day (Hainan winter) to investigate the heading date. The planting locations were Changping Station (Beijing 40.17 N. 116.23 E, long-day conditions) and Hainan Station, Hainan Province, China (Sanya, 18.30 N, 109.30 E, short-day conditions) of the Institute of Crop Sciences (ICS, CAAS), Academy of Agricultural Sciences, Beijing, China. Field management was carried out according to standard management. The heading date (DTH) of rice was the number of days from sowing to 50% tiller heading. All parents and hybrid Fi generations were designed with a randomized block experiment for field layout, with at least three repetitions.

I. Heading date and yield phenotype of DEH229 and its progeny

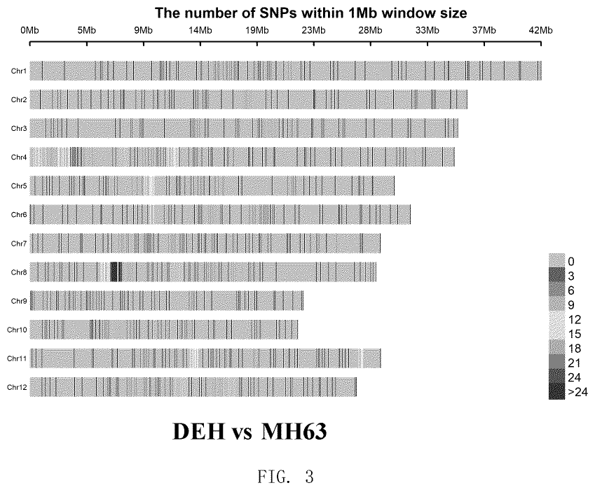

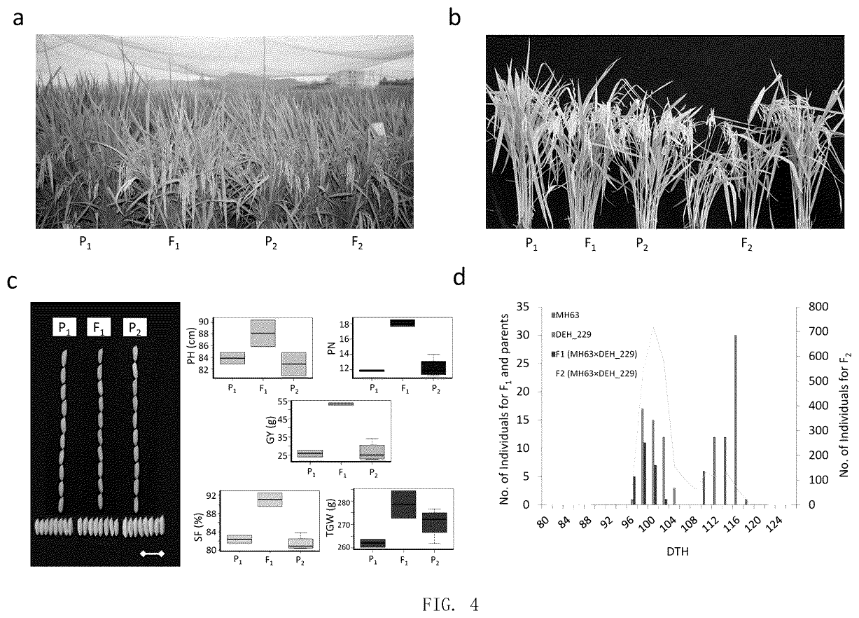

Compared with MH63, DEH229 showed carly-heading under both long-day (LD) and short-day (SD) conditions (refer to FIG. 2), and the heading date was 6 days (LD) and 12 days (SD) carlier than that of MH63, respectively, and its yield was not significantly different from that of MH63. The genome-wide SNP difference between DEH229 and the parent MH63 was only 1.06%, but its variation spread throughout the genome (FIG. 3).

The following hybrid combinations were assembled with DEH229 as the parent and middle indica restorer lines:

The hybrid combination MH63/DEH229 was assembled by using MH63 as the female parent and DEH229 as the male parent.

The hybrid combination 9311/DEH229 was assembled by using 9311 as the female parent and DEH229 as the male parent.

The hybrid combination R498/DEH229 was assembled by using R498 as the female parent and DEH229 as the male parent.

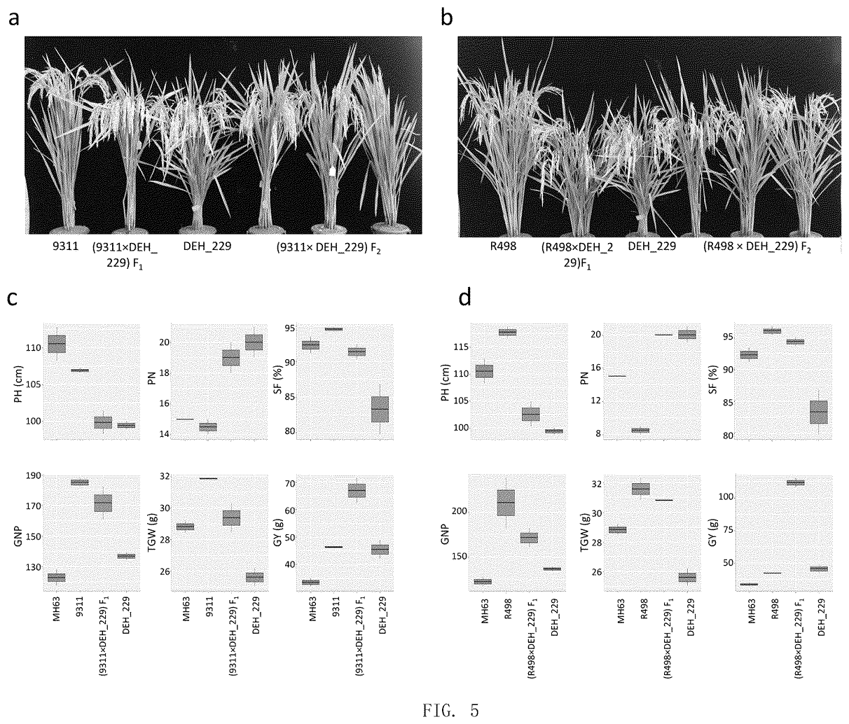

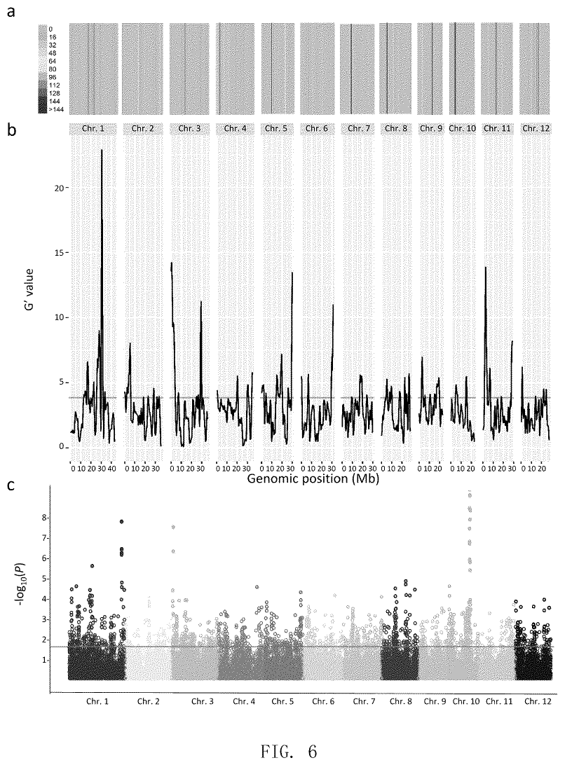

In 2018 and 2019, under short-day conditions, the performance of the hybrid progenies (F₁ and F₂) of the above-mentioned hybrid combination of DEH229 and the middle indica restorer line was continuously observed. The results showed that the F₁ generations of DEH229, MH63, 9311 and R498 not only exhibited carly-heading but also showed heterosis in yield, as shown in FIG. 4 and FIG. 5 for details:

FIG. 4 shows that the hybrid F₁ generation of MH63/DEH229 has obvious carly-heading characteristics and super-parental dominance in grain yield. Compared with the parents MH63 and DEH229, the F₁ of MH63/DEH229 showed a significant increase in effective panicle number (PN) and seed setting rate (SF), but there was no significant difference in grain shape. We investigated the dominant carly-heading phenotype and found that the dominant carly-heading trait showed a bimodal distribution. The DTH values of the main part of the F₁ and F₂ generation populations all fell within the range of DEH229, and the DTH values for the minor part of the F₂ population overlapped with the range of values of MH63. However, the ratio of carly-heading (DEH229 type) to late-heading (MH63 type) was seriously deviated from 3:1. This indicated that the carly-heading traits of DEH229 and its progeny probably involved a relatively complex genetic mechanism, but the existence of the main gene could not be ruled out. FIG. 5 showed that the yields of the F₁ generation of hybrid combinations 9311/DEH229 and R498/DEH229 were also significantly higher than those of both parents, and other characteristics of F1 were intermediate types between the parents, and the performance was similar to that of MH63/DEH229.

II. Analysis of the genetic mechanism controlling the carly-heading traits in DEH229

In order to analyze the genetic mechanism controlling carly-heading traits in DEH229, 454 individual plants with the carly-heading phenotype of DEH229 and 222 individual plants with the late-heading phenotype of MH63 were selected from the F₂ population of MH63/DEH229 (6705 individual plants in total) based on the parental, F₁ and F₂ phenotypes of the MH63/DEH229 hybrid combination under short-day conditions, and their leaves were combined in equal numbers into a mutant pool (Mut-Pool) and a wild-type pool (WT-Pool), respectively, and genomic DNA was extracted: all of the above individual plants were labeled and seeds were harvested in separate plants, with approximately 15 seeds taken from each seedling for germination, after the seedlings had grown three leaves, leaf samples were taken and then an equal weight of leaf samples from each individual plants in the same pool were mixed, and the leaf samples from the wild and mutant pools and the parents were used to extract DNA using the standard method of CTAB. A 200bp library was prepared using the Illumina Truseq DNA library protocol (Illumina Kit FC-121-4001; Illumina Inc., San 72 Diego, CA, USA) and the peak insert size was approximately 200bp. Library quality was checked using standard methods with the Agilent 2100 Bioanalyzer High Sensitivity Kit. After library analysis, libraries were sequenced by next-generation sequencing (NGS) using the Illumina HiSeq X10 platform (Illumina Inc.) by using a 150bp paired-end strategy. Localization analysis was completed using QTLseq analysis and a total of 57 loci were detected at the default threshold (figure a in FIG. 6). Next, GWAS analysis was performed using the heading date data of 3K sequencing germplasm (3K-RG) collected in Hainan in the same season; then the GWAS analysis results were combined with the above QTLseq results to select loci that were consistent between the two results, the results were shown in figure b in FIG. 6, and the number of QTLs related to heading date was reduced from 57 to 25 (61.4%) loci. The mean G′ value for the 25 loci was 5.3 (range 3.9-11.8), and the mean value for-LOG10 (P) was 2.8 (range 2.0-6.7). Of these, the top three loci for G′ values were qEH_3a (10.1), qEH_3b (9.7), and qEH_5c (11.8). Of these three, qEH_3a also had the highest-LOG10 (P) value (6.7).

For further analysis, the SNPs within the above 25 QTL regions were divided into 44SNP clusters according to an empirical threshold of about 200 kb. Haplotype analysis using RFGB v2.0 showed that 32 (72.7%) SNP clusters were significantly associated with the heading date in 3K-RG. Among these 32 SNP clusters, there was a region detected jointly by QTLseq, GWAS, and gene annotation information provided by the SNP&InDel module of RFGB v2.0,namely EH_9 within qEH_3a.

A total of 6 candidate genes related to rice heading date were found in the EH_9 interval range, as shown in Table 1 for details.

TABLE 1
Comparison of EH_9 gene region variation
in DEH229 and MH63 using HD data from RFGB

| | 3K-RG | | | | | | |
|---|---|---|---|---|---|---|---|
| | Genome | | | | 2 k [symbol] | 1500 b [symbol] | DEH22vs |
| Gene | sequence | CDS | 5′UTR | 3′UTR | promoter | promoter | MH63 |
| Os03g0112700 | * | NS | / | * | * | * | no Var |
| Os03g0122500 | * | — | — | — | NS | * | no Var |
| Os03g0122600 | * | * | * | * | * | * | 6 Var (2 |
| | | | | | | | SNP + 4 |
| | | | | | | | InDel) |
| Os03g0131900 | * | NS | / | * | * | * | no Var |
| Os03g0151300 | * | * | NS | NS | — | * | no Var |
| Os03g0169600 | * | * | * | * | * | * | no Var |

Note:
* indicates significance analysis at P < 0.05; NS indicates not significant; / indicates no SNP; — indicates data not available, no Var indicates neither SNP nor InDel variation. [symbol]
indicates data missing or illegible when filed

Comparing genomic differences between DEH229 and MH63, only two SNPs and four InDels were found within the Os03g0122600 genomic interval. There were no polymorphisms between DEH229 and MH63 at the genomic sequence level for the other five candidate genes.

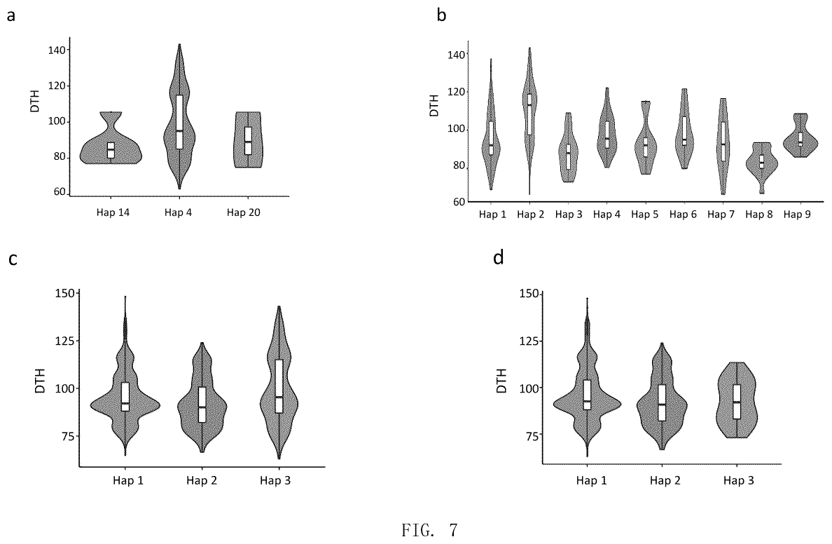

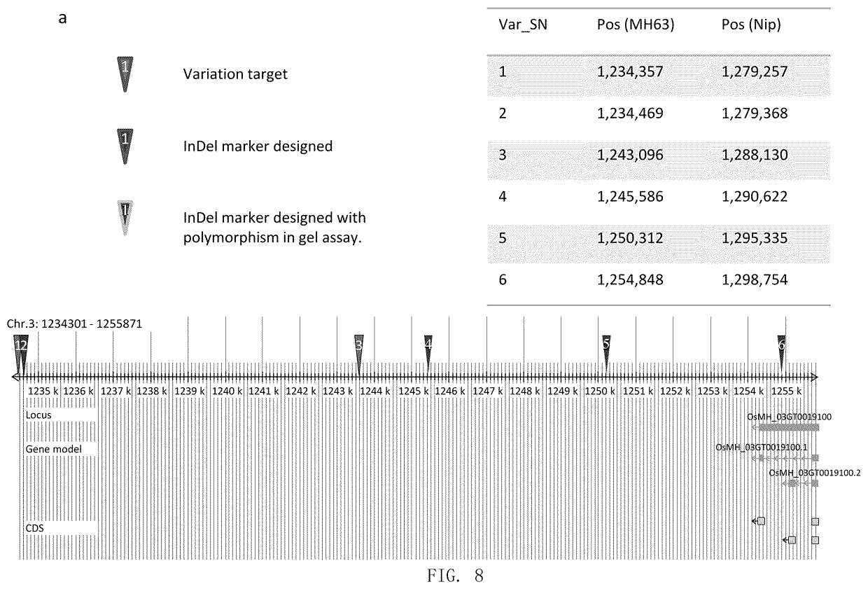

Os03g0122600 located between 1.14-1.27 Mb in EH_9 was related to the heading date of rice. The haplotype analysis map of the gene Os03g0122600 was shown in FIG. 7: three haplotypes were identified in the CDS region. Among the three haplotypes, the DTH of Hap14was the shortest compared with other haplotypes (figure a in FIG. 7). In the promoter region, a total of nine haplotypes were found in 3K-RG, among which Hap1, Hap3 and Hap8 represented shortened DTH (figure b in FIG. 7). Both 5′UTR and 3′UTR contained three haplotypes. Among the 5′UTR and 3′UTR, Hap2 had the shortest DTH (figures c and d in FIG. 7).

Notably, there were genomic differences around the DTH3 region between the reference map IRGSP1.0 and MH63RS2. In IRGSP1.0, all six variants were located within genomic regions. However, in MH63RS2, most of the six variants were found to be located in intergenic regions where no genes were annotated. In addition, according to the RIGW database, Os03g0122600 (DTH3, LOC_Os03g03100) had no corresponding annotation in MH63RS2, nor docs OsMH_03G0019100 in IRGSP1.0. This strongly suggested that the complex molecular mechanism may not be just a new allele of DTH3, but also the DEH phenotypic variation that the EH_9 region of DEH229 acted on.

3. Screening and effect verification of molecular markers

Molecular markers were designed for the four InDels found in Os03g0122600, and a total of eight pairs of primers were designed using the parental sequence as a reference to verify the possible function of the variation in Os03g0122600 on the early-heading phenotype of DEH229.

Parents MH63 and DEH229 were used as controls. In the F2 population of the hybrid progenies of MH63/DEH229 planted under short-day conditions (2017 winter, Hainan Station, Hainan Province, China (Sanya, 18.30 N. 109.30 E)), 60 carly-heading individual plants were randomly selected as near-isogenic lines of DEH229 and 60 late-heading individual plants were selected as near-isogenic lines of MH63. The progenies were derived by the single grain transmission. These 120 individual plants progenies F3 and F4 were planted at the Hainan Experimental Station (Sanya, 18.30 N, 109.30 E) in winter 2018 (short-day conditions) and Changping Experimental Station (40.17 N. 116.23 E) in summer 2019 (long-day conditions) of the Institute of Crop Sciences, Chinese Academy of Agricultural Sciences (ICS, CAAS) with the parents as a control to investigate the heading date, and the results showed a stable heading date phenotype regardless of short-day or long-day conditions. Since 11 late-heading seeds showed a low seed-setting rate under long-day conditions in 2019, they were excluded from the marking experiment. Finally, 109 lines (including 60 carly-heading lines and 49 late-heading lines) in the F₄ generation were further analyzed for markers.

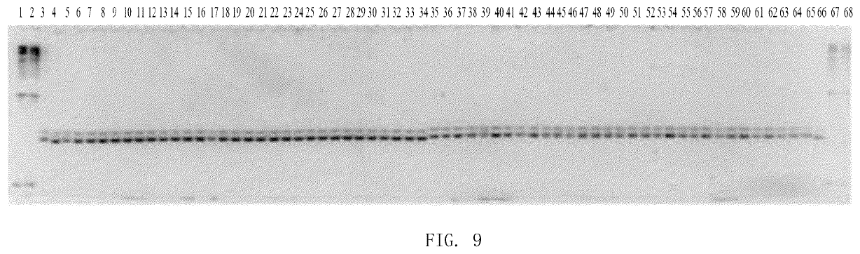

Eight pairs of primers designed above were used to carry out PCR on 109 strains respectively. PCR amplification and polyacrylamide (PAGE) gel electrophoresis were performed according to routine protocols with the following settings: initial denaturation at 95° C. for 3 min, denaturation at 95°° C. for 30 s, annealing at 57° C. for 30 s, and extension at 72° C. for 35 seconds. A total of 40 cycles were performed. PCR was followed by gel assay. The results of gel electrophoresis showed that the primer pair ZMEH_1 (ZMEH_1_F (SEQ ID No.1)/ZMEH_1_R (SEQ ID No.2)) performed the best, and part of its electropherogram was shown in FIG. 9. The specific primer pair ZMEH_1 and the targeted InDels were shown in Table 2, ATTT/AT in the table referred to the 1234469-1234472 position of the wild-type MH63 was ATTT, and that of DEH229 was AT, that is, the two bases TT of 1234471-1234472 were missing.

The PCR amplification products of MH63 and DEH229 were sequenced by conventional sequencing methods respectively, and the obtained sequence information was as follows:

MH63 of late-heading
(SEQ ID No. 3)
5′-TCTCTTGGTTTATGGGTATTAAATAACATAATTTTTTTTTTGAT
CAATAATTAGTTATCCTTGAGCAATATACGACATAAAGTAAGGTTCT
AGCAAGGGTA-3′
DEH229 of early-heading
(SEQ ID NO. 4)
5′-TCTCTTGGTTTATGGGTATTAAATAACATAATTTTTTTTGATCA
ATAATTAGTTATCCTTGAGCAATATACGACATAAAGTAAGGTTCTAG
CAAGGGTA-3′

The sequence characteristics of the amplified products in the late-heading parent MH63and the early-heading parent DEH229 using ZMEH_1 primers differed by 2 T bases, which was consistent with the original intention of the primer design.

TABLE 2
Primers used to detect the sequence variation
of Os03g0122600 between DEH229 and MH63.

| | Sequence | Variation | | |
|---|---|---|---|---|
| Primer | (5′-3′) | No. | Position | Variation |
| ZMEH_ | TCACATTA | 2 | 1,234,469 | ATTT/AT |
| 1_F | GTGTTGGA | | | |
| | GTAATAGC | | | |
| | C | | | |
| ZMEH_ | CACCATAC | | | |
| 1_R | CCTTGCTA | | | |
| | GAACCT | | | |

The MH63 PCR product of the late-heading was a band of 101bp in size, named band type A; the DEH229 PCR product of the early-heading was a band of 99bp in size, named band type B; The ratio was 2bp larger and can be resolved by gel electrophoresis. The band type of each line was the same as MH63 and marked as A, the same band type as DEH229 was marked as B, the band type was a combination of band type A and band type B and was marked as H, and the deletion and other band types were read as -.

The samples in wells 3 and 65 in FIG. 9 were MH63, and the samples in wells 4 and 66were DEH229. The samples in wells 5-64 were representative phenotypes, wherein 5-33 were early-heading lines, and the samples in wells 34-64 were late-heading strains, and the results showed that the 29 wells 5-33 were of the same band type as the DEH229 in wells 4 and 66, all of which were band type B. The 31 wells 34-64 were the same as the band type of MH63 in wells 3 and 65, all of which were band A.

The similarities and differences between the gel electrophoresis bands of the PCR amplification products and the female parent MH63 and male parent DEH229 were used to identify or predict the heading date traits of the F4 generation lines 1-109: if the gel electrophoresis band of the PCR amplification product was the same as that of MH63, the DEH229 derivative line to be identified was a candidate late-heading type; if the gel electrophoresis band of the PCR amplification product was the same as that of DEH229, the DEH229 derivative line to be identified was a candidate early-heading type. The early-heading was that the heading date under long-day conditions and short-day conditions was earlier than that of MH63 under the same conditions; there was no significant difference between the late-heading being a long-day condition or a short-day condition at heading date compared to MH63 at the heading date under the same conditions.

The present invention uses the primer pair ZMEH_1 to identify the rice heading date traits. The identification result was that the 29 lines of lines 5-33 were all early-heading rice or were candidates for early-heading date rice, and the 31 lines of lines 34-64 were all late-heading rice or candidates for late-heading rice.

The heading date of the female parent MH63, the male parent DEH229 and the F4generation lines 1-109 of MH63/DEH229 and the band type of the PCR product obtained using primer pair ZMEH_1, as well as the actual heading date statistics for each line were shown in

Table 3.

TABLE 3
Heading date and band type of PCR products obtained using
primer pair ZMEH_1 for female parent MH63, male parent DEH229
and F₄ generation lines 1-109 of MH63/DEH229

| | | Long-day heading | Short-day heading |
|---|---|---|---|
| Strain number | Band type | date (days) | date (days) |
| MH63 | Band type A | 123 | 105 |
| DEH229 | Band type B | 117 | 93 |
| 1 | Band type B | 108 | 95 |
| 2 | Band type B | 108 | 94 |
| 3 | Band type B | 101 | 94 |
| 4 | Band type B | 108 | 97 |
| 5 | Band type B | 106 | 92 |
| 6 | Band type B | 106 | 93 |
| 7 | Band type B | 108 | 97 |
| 8 | Band type B | 108 | 99 |
| 9 | Band type B | 97 | 95 |
| 10 | Band type B | 101 | 98 |
| 11 | Band type B | 108 | 97 |
| 12 | Band type B | 108 | 98 |
| 13 | Band type B | 106 | 90 |
| 14 | Band type B | 108 | 98 |
| 15 | Band type B | 106 | 97 |
| 16 | Band type B | 106 | 94 |
| 17 | Band type B | 106 | 93 |
| 18 | Band type B | 108 | 97 |
| 19 | Band type B | 106 | 95 |
| 20 | Band type B | 106 | 95 |
| 21 | Band type B | 106 | 95 |
| 22 | Band type B | 106 | 94 |
| 23 | Band type B | 104 | 93 |
| 24 | Band type B | 108 | 95 |
| 25 | Band type B | 108 | 95 |
| 26 | Band type B | 104 | 90 |
| 27 | Band type B | 102 | 92 |
| 28 | Band type B | 108 | 95 |
| 29 | Band type B | 106 | 94 |
| 30 | Band type B | 104 | 90 |
| 31 | Band type A | 120 | 113 |
| 32 | Band type A | 120 | 114 |
| 33 | Band type A | 120 | 114 |
| 34 | Band type A | 120 | 114 |
| 35 | Band type A | 122 | 116 |
| 36 | Band type A | 120 | 114 |
| 37 | Band type A | 120 | 113 |
| 38 | Band type A | 120 | 114 |
| 39 | Band type A | 120 | 113 |
| 40 | Band type A | 120 | 114 |
| 41 | Band type A | 120 | 114 |
| 42 | Band type A | 125 | 120 |
| 43 | Band type A | 120 | 113 |
| 44 | Band type A | 120 | 114 |
| 45 | Band type A | 120 | 113 |
| 46 | Band type A | 118 | 113 |
| 47 | Band type A | 118 | 114 |
| 48 | Band type A | 118 | 113 |
| 49 | Band type A | 118 | 114 |
| 50 | Band type A | 118 | 114 |
| 51 | Band type A | 120 | 115 |
| 52 | Band type A | 120 | 115 |
| 53 | Band type A | 118 | 113 |
| 54 | Band type A | 118 | 114 |
| 55 | Band type A | 120 | 114 |
| 56 | Band type A | 122 | 115 |
| 57 | Band type A | 118 | 115 |
| 58 | Band type A | 120 | 115 |
| 59 | Band type A | 124 | 117 |
| 60 | Band type A | 122 | 115 |

Statistical data in Table 3 showed that ZMEH_1 was able to explain about 86.0% of the phenotypic variation in these 109 F₄ progeny from the MH63/DEH229 population under long-day conditions, i.e. the identification of rice heading date traits using primer pair ZMEH_1was in 86.0% agreement with the identification results of heading date in the field. This indicated that primer pair ZMEH_1 could be used to identify or assist in identifying rice heading date.

The present invention has been described in detail above. For those skilled in the art, without departing from the spirit and scope of the present invention and without unnecessary experiments, the present invention can be practiced in a wider range under equivalent parameters, concentrations, and conditions. While specific embodiments of the present invention have been shown, it should be understood that the present invention can be further modified. In a word, according to the principles of the present invention, this application intends to include any changes, uses or improvements to the present invention, including changes made by using conventional techniques known in the art and departing from the disclosed scope of the present application. Applications of some of the essential features were possible within the scope of the appended claims below.

### Industrial Application

The present invention discloses a specific primer ZMEH_1 for identifying or assisting in identifying the heading date of rice materials and use thereof, and a breeding method of rice cultivars with early-heading without yield drag. The ZMEH_1 is a pair of PCR primers corresponding to the upstream and downstream of the fragment with sequence variation 1 of the rice Os03g0122600 gene between the late-heading parent MH63 and the early-heading parent DEH229. ZMEH_1 can explain about 86.0% of the phenotypic variation of the progeny from the MH63/DEH229 population, which meets the needs of industrial applications. DEH229 has the characteristic of early-heading without yield drag, and its progenies derived from various rice cultivars all continue to have the characteristic of early-heading without yield drag. The specific primer ZMEH_1 can be used to quickly screen out the materials with early-heading characteristics among the progeny rice materials derived from DEH229, and carry out rapid breeding and utilization to promote the breeding process of early-maturing rice without affecting the yield.

1-12. (canceled)

13. Any of the following substances:
(1) a specific primer for identifying or assisting in identifying the heading date of rice;
(2) a reagent or kit for identifying or assisting in identifying the heading date of rice.

14. The substance according to claim 13, characterized in that, the specific primer is two single-stranded DNAs with nucleotide sequences of SEQ ID No.1 and SEQ ID No.2 respectively.

15. The substance according to claim 13, characterized in that, the reagent or kit includes the specific primer for identifying or assisting in identifying the heading date of rice.

16. Any of the following methods:
a method for identifying or assisting in identifying the heading date of rice;
a method for rice breeding;
a breeding method of rice with early-heading without yield drag.

17. The method according to claim 16, characterized in that, the method for identifying or assisting in identifying the heading date of ricecomprising the following steps of: using the genomic DNAs of a late-heading parent MH63, an early-heading parent DEH229 and a progeny line to be identified as templates respectively, using PCR primer pairs that are formed by two single-stranded DNAs shown in SEQ ID No.1 and SEQ ID No. 2 to carry out PCR amplification, detecting an obtained PCR product, and determining the heading date of the progeny line to be identified according to the following method:
if a PCR amplification product of the progeny line to be identified is the same as that of a PCR amplification product of the late-heading parent MH63, then the progeny line to be identified is a late-heading line or a candidate for late-heading line; if the PCR amplification product of the progeny line to be identified is the same as that of an early-heading parent DEH229, the line to be identified is an early-heading line or a candidate for an early-heading line.

18. The method according to claim 17, characterized in that, the step of detecting an obtained PCR product is to detect a size of the PCR product by electrophoresis and/or to detect the sequence information of the PCR product by sequencing.

19. The method according to claim 17, characterized in that, the PCR amplification product of the late-heading parent MH63 is a single-stranded DNA with a nucleotide sequence as shown in SEQ ID No.3; the PCR amplification product of the early-heading parent DEH229 is a single-stranded DNA with a nucleotide sequence as shown in SEQ ID No.4.

20. The method according to claim 16, characterized in that, the method for identifying or assisting in identifying the heading date of rice uses any of the following substances:
(1) a specific primer for identifying or assisting in identifying the heading date of rice;
(2) a reagent or kit for identifying or assisting in identifying the heading date of rice.

21. The method according to claim 16, characterized in that, the method for rice breeding uses a specific primer for identifying or assisting in identifying the heading date of rice; or a reagent or kit for identifying or assisting in identifying the heading date of rice.

22. The method according to claim 16, characterized in that, the breeding method of rice with early-heading without yield drag comprising the following steps of: using DEH229 as a parent or one of the parents to breed progenies, using a specific primer for identifying or assisting in identifying the heading date of rice to select early-heading materials from the progenies for further breeding to obtain rice with early-heading without yield drag.

23. A rice with early-heading without yield drag produced by the method according to claim 22.
